(19) United States
(12) Patent Application Publication
(10) Pub. No.: US 2010/0179429 A1
(43) Pub. Date: Jul. 15, 2010

(54) HANDHELD VOLUMETRIC ULTRASOUND SCANNING

(76) Inventors: Ronald HO, Mercer Island, WA (US); Shih-Ping Wang, Los Altos, CA (US)

Correspondence Address: COOPER & DUNHAM, LLP, 30 Rockefeller Plaza, 20th Floor, NEW YORK, NY, 10112, US

(21) Appl. No.: 12/691,915

(22) Filed: Jan. 22, 2010

Related U.S. Application Data
(63) Continuation-in-part of application No. 12/238,091, filed on Sep. 25, 2008.
(60) Provisional application No. 61/147,358, filed on Jan. 26, 2009.
(60) Provisional application No. 61/081,204, filed on Jul. 16, 2008.

(51) Int. Cl. A61B 8/14

(52) U.S. Cl. 600/446

(57) ABSTRACT

Systems, methods, and related computer program products for ultrasonic examination of a tissue volume, such as a human breast, are described. A handheld volumetric ultrasound scanning probe, which is characterized by a two-dimensional scan area with a substantially rigid cap extending across the two-dimensional scan area, is provided with a texturably couplant-porous material sheet covering the substantially rigid cap over at least a portion of the two-dimensional scan area. The texturably couplant-porous material sheet facilitates positional stability of the handheld volumetric ultrasound scanning probe while positioned against a skin surface of the tissue volume. Advantageously, while the texturably couplant-porous material sheet brings about a more stable physical interface at the skin surface, the texturably couplant-porous material sheet brings about little or no degradation in acquired image quality. Also described are systems and related methods for elastography imaging using the handheld volumetric ultrasound scanning probe.

### CROSS-REFERENCE TO RELATED APPLICATIONS

This patent application claims the benefit of U.S. Provisional Ser. No. 61/147,358, filed Jan. 26, 2009. This patent application is a continuation-in-part of U.S. Ser. No. 12/238,091, filed Sep. 25, 2008, which claims the benefit of U.S. Provisional Ser. No. 61/081,204 filed Jul. 16, 2008 and which published as US2009/0024039A1 on Jan. 22, 2009. Said applications Ser. Nos. 61/147,358, 12/238,091 and 61/081,204 are hereby incorporated by reference herein.

### FIELD

This patent specification relates to the ultrasonic imaging of biological tissues. More particularly, this patent specification relates to a handheld volumetric ultrasound scanning device that is advantageously usable in a variety of ultrasound imaging contexts including, but not limited to, freehand ultrasound elastography imaging of the breast.

### BACKGROUND AND SUMMARY

FIG. 1 illustrates a side view of a handheld volumetric ultrasound scanning probe 102 according to the prior art, comprising a casing 104 within which a linear array transducer 106 is mechanically swept back and forth in a periodic fashion to acquire volumetric images of biological tissue. The handheld volumetric ultrasound scanning probe 102, which is sometimes termed a wobbler probe, is characterized by a two-dimensional scan area 112 representing the area scanned as the device is held against (and sufficiently compressed against) the skin surface of the tissue volume. For so-called wobbler probes, the two-dimensional scan area is generally coextensive with the length of the linear array transducer therein times its mechanical range of movement therein. For other types of handheld volumetric ultrasound scanning probes such as those containing fixed solid-state two-dimensional ultrasound transducers, the two-dimensional scan area is generally coextensive with the extent of those two-dimensional ultrasound transducers across the inside of the casing. The casing 104, which includes a base portion 108 and a lid portion 110, typically comprises a rigid plastic material such as acrylonitrile butadiene styrene (ABS) plastic. The handheld volumetric ultrasound scanning probe 102 is further characterized by a substantially rigid cap 114 that extends across the two-dimensional scan area 112. In the example shown, the cap 114 is integral with the lid portion 110, its lateral extent corresponding to the lateral extent of the two-dimensional scan area 112.

FIG. 2A illustrates a perspective exploded view of the handheld volumetric ultrasound scanning probe 102 with the lid portion 110 removed from the base portion 108 to expose the linear array transducer 106 and its related driving mechanism. The linear array transducer 106 can typically be translated at a rate of between 0.5-30 sweeps per second to provide real-time 3D imaging (“4D” imaging) of the underlying biological tissue. FIG. 2B illustrates the handheld volumetric ultrasound scanning probe 102 with the lid portion 110 of the casing 104 properly sealed to the base portion 108.

One issue that arises with handheld volumetric ultrasound scanning probe 102 relates to a slippery, positionally unstable contact between the outer surface of the cap 114 and the skin surface of the underlying biological tissue, this positionally unstable contact being brought about by a unique combination of factors. First, according to the known prior art, the cap 114 has a smooth outer surface in order to inhibit unwanted scattering of the acoustic pulses generated and received by the linear array transducer 106 and the associated distortions or artifacts that would appear in the resultant images. Second, an ultrasound couplant comprising a slippery liquid or gel is typically applied where the cap 114 meets the skin surface in order to facilitate ultrasound coupling and good image quality. Third, in keeping with its essential purpose, the scan area 112 covered by the cap 114 extends over a relatively large area of skin, typically 2 cm×2 cm or 3 cm×3 cm or greater. This relatively large footprint exacerbates the effects of the surface smoothness and the couplant to result in an interface that is substantially more slippery and unstable, for example, than that of a conventional handheld linear array probe having a relatively narrow footprint over the skin. The high degree of slipperiness and instability must be compensated for by the skill and effort of the clinician to avoid unwanted translation over the skin when imaging a particular volume for any appreciable period of time.

The inconvenience of a slippery, unstable interface at the skin surface becomes particularly problematic when the handheld volumetric ultrasound scanning probe 102 is used in the context of freehand ultrasound elastography imaging. Ultrasound elastography refers to the investigation of tissue elasticity using ultrasound methods, wherein an externally-induced stress is applied to a tissue sample and the resulting strain of the tissue sample is determined from the change in the ultrasound echo readings before and after the application of the stress. With ultrasound elastography, a sample of tissue can be characterized by mapping out the local elastic strain of the analyzed tissue, the resulting mapping often being termed an elastogram. Typically, hard tumor material will show less strain than softer tissues and this contrast can be picked up in the elastogram. In freehand ultrasound elastography, the same handheld ultrasound probe is used to (i) acquire the pre-compression images, (ii) compress the tissue sample by transfer of downward/inward force from the clinician's hand to the skin surface, and (iii) acquire the post-compression images.

The presence of a slippery, unstable interface at the skin surface makes the process of freehand ultrasound elastography imaging particularly difficult when using the handheld volumetric ultrasound scanning probe 102 of FIGS. 1-26. When the body part is the breast, this process becomes even more difficult because of the pendulous, unwieldy nature of the breast.

Accordingly, it would be desirable to provide a handheld volumetric ultrasound scanning probe that exhibits a more stable interface at the skin surface while at the same time providing little or no degradation in acquired image quality. It would be further desirable to provide such a handheld volumetric ultrasound scanning probe in a manner that does not contribute substantially to its overall cost. It would be even further desirable to provide retrofitted and/or adapted versions of existing handheld volumetric ultrasound scanning probes to exhibit such increased stability at the skin interface. It is to be appreciated that although particularly advantageous in the context of the so-called wobbler probe of FIGS. 1-2B, the preferred embodiments described infra can be advantageously applied in the context of any of a variety of handheld volumetric ultrasound scanning probes whose comparatively large, smooth footprints can make them slippery and unstable against the skin surface, such probes including, but not limited to, two-dimensional sparse array ultrasound transducers, other solid-state two-dimensional ultrasound transducers, and other mechanically driven (e.g., spinner, rotating-wheel, etc.) volumetric ultrasound transducers. Other issues arise as would be readily apparent to one skilled in the art in view of the present disclosure.

According to one preferred embodiment, provided is an apparatus for ultrasonically scanning a tissue volume, comprising a handheld volumetric ultrasound scanning probe characterized by a two-dimensional scan area and having a substantially rigid cap that extends across the two-dimensional scan area, wherein a texturably couplant-porous material sheet covers the substantially rigid cap over at least a portion of the two-dimensional scan area. The texturably couplant-porous material sheet facilitates positional stability of the handheld volumetric ultrasound scanning probe while positioned against a skin surface of the tissue volume. Advantageously, while the texturably couplant-porous material sheet brings about a more stable physical interface at the skin surface, the texturably couplant-porous material sheet brings about little or no degradation in acquired image quality.

Also provided is a method for ultrasound examination of a compressible tissue volume, comprising providing a handheld volumetric ultrasound scanning probe having a cap and a two-dimensional scan area thereacross, wherein the handheld volumetric ultrasound scanning probe is provided with a texturably couplant-porous material sheet at least partially covering the two-dimensional scan area of the cap. The handheld volumetric ultrasound scanning probe is operated to acquire ultrasonic scans of the tissue volume, by hand-manipulating the unit to bring the two-dimensional scan area of the cap, as at least partially covered by the texturably couplant-porous material sheet, into contact with a skin surface of the tissue volume. The texturably couplant-porous material sheet facilitating positional stability of the handheld volumetric ultrasound scanning probe while so positioned against the skin surface.

Also provided is a system for ultrasonic examination of a compressible tissue volume, comprising a handheld volumetric ultrasound scanning probe characterized by a two-dimensional scan area and having a substantially rigid cap that extends across the two-dimensional scan area, and a texturably couplant-porous material sheet covering the substantially rigid cap over at least a portion of the two-dimensional scan area. The texturably couplant-porous material sheet facilitates positional stability of the handheld volumetric ultrasound scanning probe while positioned against a skin surface of the tissue volume. The system further comprises a processor operatively coupled to the handheld volumetric ultrasound scanning probe. The processor receives first ultrasonic scans from the handheld volumetric ultrasound scanning probe while in a first state of compression against the skin surface, receives second ultrasonic scans from the handheld volumetric ultrasound scanning probe while in a second state of compression against the skin surface different than the first state of compression, and processes the first and second ultrasonic scans to identify a potential lesion in the tissue volume that is exhibiting different elasticity characteristics in comparison to surrounding tissue. The system further comprises an output display coupled to the processor, the output display displaying at least one ultrasonic image of the potential lesion and the surrounding tissue in a manner that visually highlights, in colorized format, the different elasticity characteristics of the potential lesion relative to the surrounding tissue.

### BRIEF DESCRIPTION OF THE DRAWINGS

FIG. 1 illustrates a side view of a handheld volumetric ultrasound scanning probe according to the prior art;

FIGS. 2A-2B illustrate perspective views of the handheld volumetric ultrasound scanning probe of FIG. 1;

FIGS. 3A-3B illustrate side views of a handheld volumetric ultrasound scanning probe according to a preferred embodiment;

FIGS. 4A-4B illustrate perspective views of the handheld volumetric ultrasound scanning probe of FIGS. 3A-3B;

FIG. 5A illustrates a side view of a handheld volumetric ultrasound scanning probe according to a preferred embodiment;

FIG. 5B illustrates a perspective view of the handheld volumetric ultrasound scanning probe of FIG. 5A;

FIGS. 6A-6B illustrate ultrasonic elastography imaging using a handheld volumetric ultrasound scanning probe according to a preferred embodiment;

FIGS. 7-10 illustrate disposable covers for a handheld volumetric ultrasound scanning probe according to one or more preferred embodiments; and

FIGS. 11A-11C illustrate ultrasound imaging of a tissue volume using a handheld volumetric ultrasound scanning probe according to one or more preferred embodiments.

### DETAILED DESCRIPTION

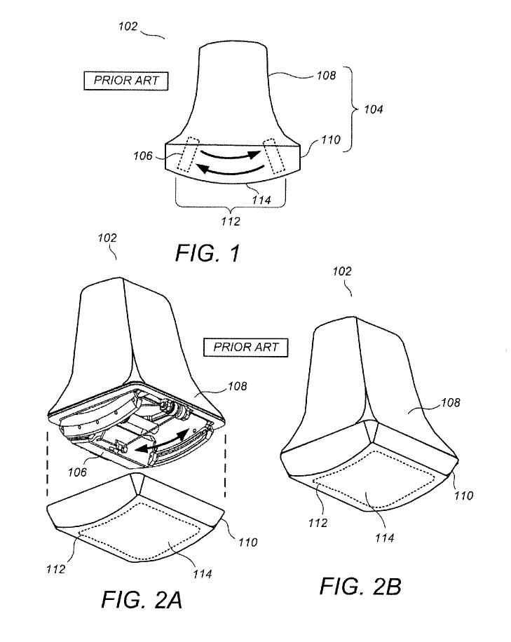

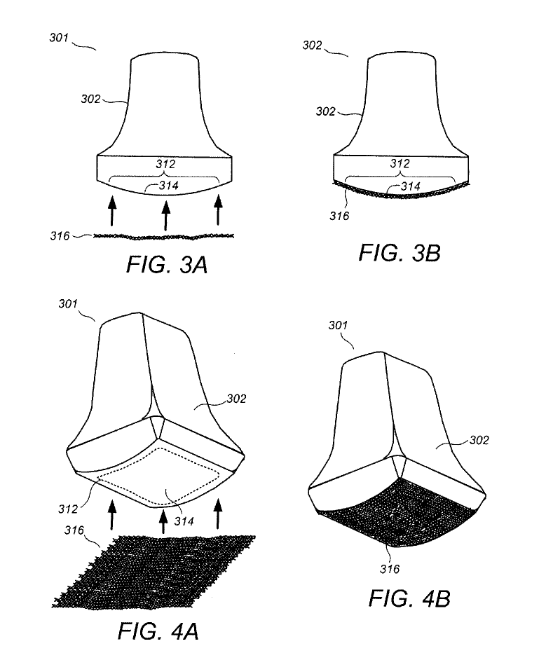

FIGS. 3A, 3B, 4A, and 4B illustrate a stabilized handheld volumetric ultrasound scanning probe 301 according to a preferred embodiment. The stabilized handheld volumetric ultrasound scanning probe 301 comprises a smooth-capped handheld volumetric ultrasound scanning probe 302 similar to the handheld volumetric ultrasound scanning probe 102 of FIG. 1, supra, including a rigid, smooth cap 314 extending over a two-dimensional scan area 312. According to a preferred embodiment, the cap 314 is at least partially covered by a texturably couplant-porous material sheet 316. It has been found that the presence of the texturably couplant-porous material sheet 316 covering the cap 314 over at least a portion of the two-dimensional scan area 312 facilitates positional stability of the handheld volumetric ultrasound scanning probe 302 over the skin surface of the tissue volume, while at the same time having negligible adverse effect on image quality when compared to the use of the smooth-capped probe by itself.

In accordance with one preferred embodiment, the texturably couplant-porous material sheet 316 can be similar to one or more of those described in the commonly assigned U.S. Ser. No. 12/238,091, supra, and the commonly assigned U.S. Prov. Ser. No. 61/081,204, supra. However, for preferred embodiments in which the texturably couplant-porous material sheet 316 comprises a membraneous material, it is not required that it be particularly taut when placed over the cap 314, because the substantial rigidity of the cap 314 is capable of providing any necessary downward compressive force against the skin, and thus there is no dependence on the tautness of a membrane for providing downward force against the skin.

For one preferred embodiment, the texturably couplant-porous material sheet 316 comprises a fabric sheet having material properties similar to those described in the commonly assigned WO2007/014292A2, which is incorporated by reference herein. As used herein, fabric refers generally to a material structure of interconnected parts, such as can be formed by knitting, weaving, or felting natural or synthetic fibers, assembling natural or synthetic fibers together into an interlocking arrangement, fusing thermoplastic fibers, or bonding natural or synthetic fibers together with a cementing medium, and further refers to materials having similar textures or qualities as those formed thereby, such as animal membranes or other naturally occurring substances having fabric-like properties (either inherently or by processing), and such as materials generated by chemical processes yielding fabric-like webbings. One suitable material for the taut fabric sheet comprises a polyester organza material having a filament diameter of about 40 microns and a filament spacing of about 500 microns. However, the taut fabric sheet may comprise any of a variety of other fabrics that are substantially inelastic and generally porous to ultrasound couplants without departing from the scope of the present teachings. Examples include, but are not limited to, polyester chiffon fabrics and cloth fabrics comprising straight weaves of substantially inelastic fibers. Where the weave is particularly tight (for example, the cloth used in men's dress shirts or the cloth used in many bed sheets), porosity can be achieved by introducing fine perforations or other irregularities that allow the ultrasound couplant to more readily soak therethrough.

As an alternative to a fabric sheet, or in combination therewith, the texturably couplant-porous material sheet 316 can comprise a vented membrane as described in WO2007/014292A2, supra, in which a membraneous material is patterned with voids therethrough. Examples of materials that can be used for the vented membrane include, but are not limited to, polypropylene, polyester (including but not limited to Mylar), polyethylene, PTFE, PET, paper, Kevlar, metal, and epoxy-fiber composite materials. Preferably, the size of the voids and the average void pitch is equal to or greater than the wavelength of the acoustic signals being applied. By way of example, for a 7 MHz ultrasound frequency, the size of the voids should be about 0.5 mm or greater. The vented membrane can be formed, for example, by beginning with a uniform film sheet and establishing a void pattern therein by one of stamping, perforating, or other process designed to establish a void pattern. Examples include laser perforation, perforation using hot needles, die cutting, cold stamping, and hot-stamping. For one preferred embodiment, the vented membrane comprises a film sheet less than 1 mm thick, with at least 25% of a surface area of the film sheet being occupied by voids. In another preferred embodiment, at least 80% of the surface area is occupied by voids. In an alternative fabrication method, the vented membrane can be formed by a vertical fusing of a first monofilamental pattern and a second monofilamental pattern. In one example, each monofilamental pattern can comprise 0.04 mm monofilaments having a pitch of about 0.5 mm.

Advantageously, the presence of the texturably couplant-porous material sheet 316 reduces slipperiness of the couplant-wetted probe-skin contact, making the device easier to manipulate in comparison to the smooth-capped probe of FIGS. 1, 2A, and 2B. Although advantageous for a variety of different volumetric ultrasound imaging procedures for a variety of different body parts, the reduced slipperiness is particularly advantageous when the handheld volumetric ultrasound scanning probe 302 is being used to apply significant pressure to the skin surface in freehand ultrasound elastography imaging of the breast. At the same time, the presence of the couplant-wetted texturably couplant-porous material sheet 316 between the cap 314 and the skin surface advantageously brings about little or no reduction in image quality in comparison to the use of a smooth-capped probe by itself. Although the precise physics might not yet be completely understood regarding the particular acoustic interactions happening at the wetted interfaces among the cap, the texturably couplant-porous material sheet, and the skin surface, it is believed that good image quality is maintained at least because the texturably couplant-porous material sheet promotes the dissipation of air bubbles at the wetted interfaces.

For one preferred embodiment, the smooth-capped handheld volumetric ultrasound scanning probe 302 is an off-the-shelf product supplied by any of a variety of different manufacturers, while the texturably couplant-porous material sheet 316 is provided as a separate element that can be attached by an end user (e.g., medical technician, radiologist, etc.) or their support staff. By way of example, the texturably couplant-porous material sheet 316 can contain a sparse, thin layer of adhesive (not shown) on the upper surface thereof that will stick when pressed against the cap 314, the user peeling off a thin protective sheet (not shown) before assembly. As another example, the texturably couplant-porous material sheet 316 can be provided as a sock-like covering, or can be provided with various clips or other means of attachment. In one preferred embodiment, the texturably couplant-porous material sheet 316 can be provided as a disposable element that is designed to (i) sufficiently adhere to the cap 314 for purposes of the ultrasound imaging procedure, while being (ii) sufficiently removable for easy removal and disposal thereafter.

In another preferred embodiment, the texturably couplant-porous material sheet 316 can be applied by the end user and/or their institution as a one-time permanent or semi-permanent modification to the smooth-capped handheld volumetric ultrasound scanning probe 302 for repeated use. In still another preferred embodiment, the texturably couplant-porous material sheet 316 can be adhered, affixed, strapped, fused, or otherwise integrated with the cap 314 at the time of fabrication of handheld volumetric ultrasound scanning probe 302, and/or at a downstream aftermarket modifying facility, with the resultant stabilized handheld volumetric ultrasound scanning probe 301 being sold as a pre-assembled product to the end user or institution.

Examples of devices that may be suitable as the handheld volumetric ultrasound scanning probe 302 from which a stabilized handheld volumetric ultrasound scanning probe 301 can be formed are discussed in US07334478B2 and US20070016060A1, each of which is incorporated by reference herein. Other examples include certain real-time 3D (“live 3D,” “4D”) ultrasound scanning probes such as the Voluson 730 probe available from General Electric Medical Systems. Still other examples include any of a variety of handheld volumetric ultrasound scanning probes whose comparatively large, smooth footprints (e.g., 2 cm×2 cm, 3 cm×3 cm, etc.) can make them slippery and unstable against the skin surface, including ones based on two-dimensional sparse array ultrasound transducers or other solid-state two-dimensional ultrasound transducers.

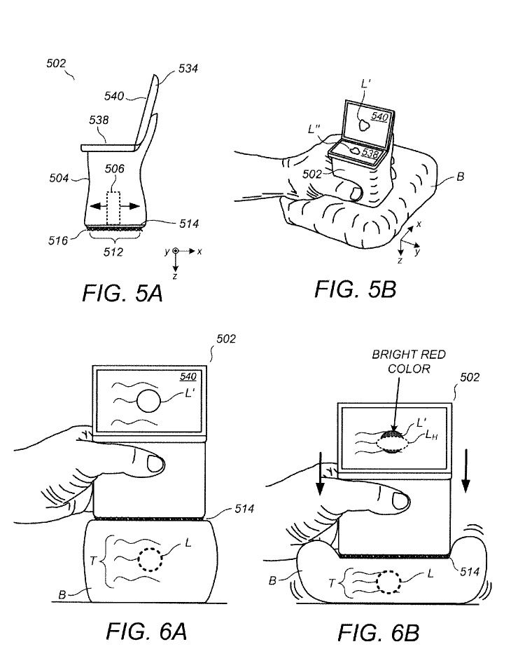

FIGS. 5A and 5B illustrate side and perspective views, respectively, of a handheld ultrasound scanning apparatus 502 according to a preferred embodiment that is particularly advantageous for use in freehand ultrasound elastography imaging. FIGS. 6A and 6B illustrate front views of the handheld ultrasound scanning apparatus 502 prior to compression and during compression, respectively, of a breast phantom B in a freehand ultrasound elastography imaging procedure according to a preferred embodiment. Handheld ultrasound scanning apparatus 502 is structurally similar to a handheld ultrasound scanning apparatus described in an ultrasound-assisted biopsy context in the commonly assigned U.S. Ser. No. 12/238,091, supra, comprising a casing 504 configured for single-handed manipulation, and further comprising a mechanically swept transducer 506 therein, except that a two-dimensional scan area 512 thereof is covered by a rigid plastic cap 514 that is, in turn, covered by a texturably couplant-porous material sheet 516 similar to the texturably couplant-porous material sheet 316 of FIGS. 3A-4B, supra. Handheld ultrasound scanning apparatus 502 further comprises a first display screen 538 and a clamshell-style lid 534 including a second display screen 540, the display screens 538 and 540 being adjacently viewable by the clinician when the lid 534 is in an open position and the clinician is manipulating the handheld ultrasound scanning apparatus 502.

In operation according to one preferred embodiment, the display screen 538 displays a two-dimensional image representative of the scanned tissue volume that would be perceived by a hypothetical acoustic impedance camera positioned directly above the handheld ultrasound scanning apparatus 502 during the procedure (i.e., positioned some distance along the negative-z axis in the coordinate system of FIG. 5B and looking downward in the positive-z direction toward the scene), wherein such hypothetical acoustic impedance camera would be able to “see” acoustic impedance, rather than light, emanating from the scanned tissue volume. Such two-dimensional image can be achieved, for example, by computing a maximum intensity projection (MIP) image in the z-direction from the acquired ultrasound volume, although other image composition methods can be used. Alternatively or in conjunction therewith, the display screen 540 displays a two-dimensional image representative of the scanned tissue volume that would be perceived from a hypothetical acoustic impedance camera positioned some distance in the negative-x direction relative to the handheld ultrasound scanning apparatus 502 and looking in the +x direction toward the scanned tissue volume scene, which can be composited in a manner similar to the image of display 538 except along the x-direction instead of the z-direction. Alternatively or in conjunction therewith, the display screen 538 may display any particular subsurface x-y plane or slabbed adjacent group of subsurface x-y planes passing through or near a subsurface lesion L, while the display screen 540 may display any particular y-z plane or slabbed adjacent group of y-z planes passing through or near the lesion L. Among other advantages, the clinician is not required to look away from the area of their hands to view the ultrasound images. In the example of FIG. 5B, the lesion L appears as a viewable lesion image L″ on the display 538 and as a viewable lesion image L′ on the display 540.

The clamshell-style lid 534 is preferably openable and closeable in a manner similar to the lids of notebook computers, flip-phones, and so forth, and may optionally be rotatable once it has been opened, as with the lids of certain notebook computers, so that the display 540 can face a different way. For one preferred embodiment, one or more of the display screens 538 and 540 can be similar to the touchscreens provided with iPhones, BlackBerries, and similar devices to allow for control inputs along with their display capabilities. For one preferred embodiment, the handheld ultrasound scanning apparatus 502 is entirely self-contained, with an on-board power source, ultrasound beanformers, processors, and controllers such that no communication with an external unit is required. For another preferred embodiment, the handheld ultrasound scanning apparatus 502 can be partially self-contained in that it comprises an onboard power source and is wirelessly connected to external processors/controllers. For still another preferred embodiment, the handheld ultrasound scanning apparatus 502 is connected by one or more electrical and/or electrooptical cables to one or more external units that provide power, control, beamforming, ultrasound processing, and display processing.

Illustrated with respect to FIGS. 6A-6B is a device and method for facilitating handheld elastography imaging according to a preferred embodiment, the method comprising identifying a potential lesion in the tissue volume that is exhibiting different elasticity characteristics in comparison to surrounding tissue, and causing an output display to visually highlight in color those different elasticity characteristics relative to the surrounding tissue. Thus, illustrated in FIG. 6A is the handheld ultrasound scanning apparatus 502 when the breast phantom B is in an uncompressed state, while FIG. 6B illustrates the handheld ultrasound scanning apparatus 502 when the breast phantom B is in a compressed state. In this particular example, the lesion L is harder than the surrounding tissue and therefore substantially keeps its shape even when the breast is compressed, whereas the surrounding tissue T experiences substantial compression when the breast is compressed. If the lesion L were of the same or similar compressibility as the surrounding tissue T, it would have taken on a the compacted shape L_{H} shown in FIG. 66. According to a preferred embodiment, the display 540 contrasts in a bright color, such as red (illustrated as dark stippling in FIG. 6B), the area occupied by the lesion that would have been compacted into the area L_{H} if the lesion was not substantially harder than the surrounding tissue T, thereby facilitating the handheld elastography imaging process by making it very easy and quick for the clinician to identify and locate these kinds of areas.

Thus, according to the preferred embodiment of FIGS. 6A-6B, when the state of compression has been changed from a first state (FIG. 6A) to a second state (FIG. 6B), there is provided on the output displays, at FIG. 6B, a second ultrasonic image in composited colorized contrast with a first ultrasonic image. The first ultrasonic image contains the potential lesion (L′) and the surrounding tissue in the second state of compression, while the second ultrasonic image contains a hypothetical version L_{H} of the potential lesion that would have appeared if that potential lesion had the same elasticity characteristic as the surrounding tissue while the tissue volume was compressed from the first state (FIG. 6A) to the second state (FIG. 6B). Notably, if the potential lesion does have the same compressibility as the surrounding tissue, then the versions L′ and L_{H} will be the same, and there will be no colorization for that lesion. Thus, the display of FIG. 6B becomes particularly rich in useful information when there are multiple potential lesions within the scanned and displayed volume, since it will be only the potential lesions having different elasticity as compared to surrounding areas that will show up as colorized on the display of FIG. 6B. In view of the present disclosure, a person skilled in the art could implement the disclosed method using known volumetric lesion segmentation techniques.

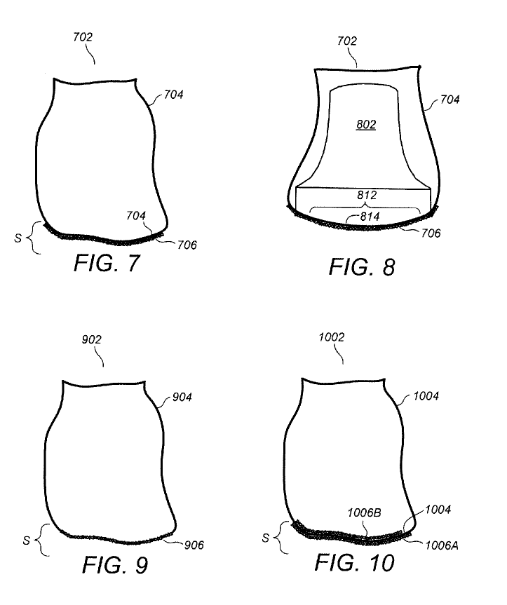

FIGS. 7-8 illustrate a disposable ultrasound probe cover 702 according to a preferred embodiment, which can be stocked at the medical site for one-time use with a conventional off-the-shelf smooth-capped handheld volumetric ultrasound probe 802 having a two-dimensional scanning area 812 and a rigid cap 814. The disposable ultrasound probe cover 702 comprises a thin, conformable, minimally attenuating material layer 704 formed in a bag-like shape to which is affixed a texturably couplant-porous material sheet 706 similar to the texturably couplant-porous material sheet 316 of FIG. 3A, supra, at locations that will at least partially cover the two-dimensional scanning area 812. For one preferred embodiment the thin, conformable, minimally attenuating material layer 704 comprises a PVDC-based (polyvinylidene chloride-based) material similar to Saran™ wrap, although this preferred embodiment may be slightly less advantageous because both the inside and the outside of the disposable ultrasound probe cover 702 will require wetting with couplant. In another preferred embodiment (not shown), the entire probe cover consists solely of the texturably couplant-porous material sheet shaped into a bag-like or sock-like shape.

FIG. 9 illustrates a disposable ultrasound probe cover 902 according to a preferred embodiment that is similar to the disposable ultrasound probe cover 702 of FIG. 7, except that a texturably couplant-porous material sheet 906 is positioned across an opening in a thin, conformable, minimally attenuating material layer 904 such that the texturably couplant-porous material sheet 906 will directly contact the smooth probe cap. FIG. 10 illustrates a disposable ultrasound probe cover 1002 according to yet another preferred embodiment, comprising a thin, conformable, minimally attenuating material layer 1004 to which is attached a first texturably couplant-porous material sheet 1006A on the outside and a second texturably couplant-porous material sheet 1006B on the inside.

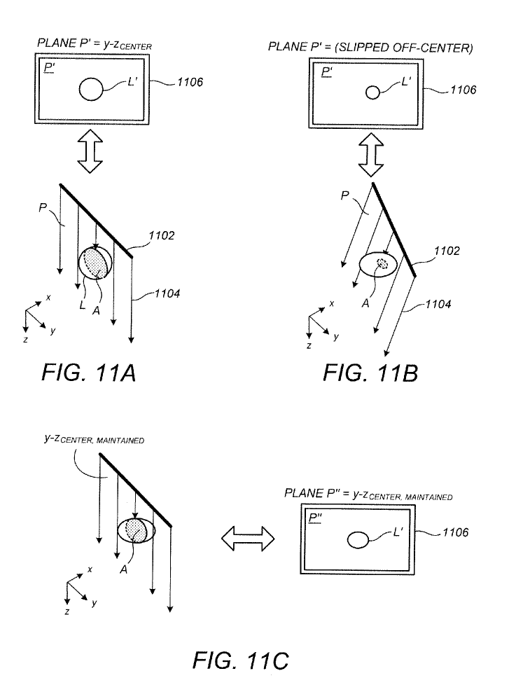

FIGS. 11A-B illustrate a visualization problem that can arise due to unintended probe movements (e.g., slippage) in freehand ultrasound elastography imaging, or other types of volumetric ultrasound imaging, based on a handheld volumetric ultrasound scanning probe. FIG. 11C illustrates a method for processing and displaying ultrasound information acquired using a handheld volumetric ultrasound scanning probe according to a preferred embodiment. As described supra, the problem of probe slippage can be substantially obviated by using a handheld volumetric ultrasound scanning probe having a texturably couplant-porous material sheet covering its cap according to one or more of the preferred embodiments supra. However, in the event there is other unintended probe movements, the preferred embodiment of FIG. 11C can be employed to ameliorate the adverse effects illustrated in FIGS. 11A-B. Alternatively, the preferred embodiment of FIG. 11C can be employed to ameliorate the adverse effects illustrated in FIGS. 11A-B in the event a smooth-capped probe is used.

It is often the case that the user will desire to monitor a planar ultrasound image P′ corresponding to single plane P passing through the center of a lesion L, as illustrated in FIG. 11A, as they are applying different amounts of pressure (or, in other applications, taking a biopsy sample or doing some other diagnostic evaluation). When a handheld volumetric ultrasound probe having an oscillated linear transducer is used, this can be achieved by either (i) freezing the movement of the linear array probe at a single location 1102 and using standard 2D scanning to scan along lines 1104 defining the plane P, or (ii) operating in 3D wobbler mode and extracting the image P′ from the resultant image volume along the plane P, wherein plane P is in fixed relationship to the probe's own coordinate system. Shown in FIG. 11A is the image P′ on a display 1006 including a lesion image L′ corresponding to the planar locus A of the lesion L that is intersected by the plane P. Prior to compression, the user will generally position the handheld volumetric ultrasound probe to an optimal location where the plane P cuts through the middle or centroid of the lesion L, making the image L′ appear at its largest. Compression is then applied. Problematically, if the relative position of the handheld volumetric ultrasound probe and the lesion L is perturbed (see FIG. 11B) due to the compressive motion or other factors, the planar locus A can shift relative to the lesion L. Problematically, the user will not be sure whether any changes in the appearance of the lesion L′ (see FIG. 11B) were due to the compression or to the slippage, thus making the evaluation more difficult.

According to a preferred embodiment, as illustrated in FIG. 11C, the handheld volumetric ultrasound probe is operated in 3D wobbler mode and the image volume is processed in real-time such that an image plane P″ is displayed, wherein the image plane P″ is maintained in real-time to correspond to an optimal plane (denoted as y-z_{CENTER,MAINTAINED}) that passes through the lesion L. In this way, the user can visually achieve an “apples to apples” comparison of the pre-compression and post-compression images in real-time during the procedure. In other preferred embodiments, additional planar views can be shown on the display 1106 or on nearby displays to show multiple orthogonal planes passing through the optimal center or centroid of the segmented lesion L. In view of the present disclosure, a person skilled in the art could implement the disclosed methods using known volumetric lesion segmentation, centroid evaluation, and coordinate-shifting techniques.

Thus, according to one preferred embodiment, the problem of “out of plane” errors is further obviated using the steps of: (i) acquiring a pre-compression 3D volume; (ii) identifying and segmenting a lesion in the pre-compression 3D volume; (iii) acquiring a post-compression 3D volume; (iv) identifying and segmenting that same lesion in the post-compression 3D volume; (v) comparing the segmented shapes of the pre-compressed and post-compressed lesions; and (vi) displaying a 2D elastography image in a manner that preserves the integrity of a subject plane passing through the lesion.

According to another preferred embodiment, a special user interface display associated with the above 3D technique can be invoked using one or more special display-mode buttons integral with the handheld wobbler probe, wherein: (vii) in a first mode, the user display shows a regular 2D view of a plane that is fixed relative to the probe; (viii) in a second mode, the user display shows three specially processed, orthogonal, lesion-centric 2D elastography images, each 2D elastography image corresponding to a respective one of three (3) orthogonal planes (x-y plane, y-z plane, and x-z plane) passing through a computed centroid (or other optimal center location) of the lesion in its compressed state as registered to a computed centroid (or other optimal center location) of the lesion in its uncompressed state, each 2D elastography image containing a display similar to that of FIG. 6B in which an image of the compressed version of the lesion is overlaid with a highlighted color outline of the uncompressed version of the lesion. Advantageously, these orthogonal views will be more robust against probe slippage or other unwanted probe movements, because their computation and display is based upon something that maintains its integrity against such variations. Optionally, in a third mode, other useful views can be shown, such as a perspective rendering of the compressed lesion adjacent to a perspective rendering of the uncompressed lesion, a color-coded animation showing a perspective rendering of the uncompressed lesion “morphing” into a perspective rendering of the compressed lesion, and so forth.

Whereas many alterations and modifications of the present invention will no doubt become apparent to a person of ordinary skill in the art after having read the foregoing description, it is to be understood that the particular embodiments shown and described by way of illustration are in no way intended to be considered limiting. By way of example, although particularly advantageous in the context of freehand ultrasound elastography imaging of the breast, a stabilized handheld volumetric ultrasound scanning probe according to the preferred embodiments can be advantageously used in a variety of different ultrasound imaging contexts including, but not limited to: freehand biopsy of the breast or other body parts, radiation force elastography of the breast or other body parts; vibrational Doppler elastography of the breast or other body parts; miscellaneous volumetric imaging of the breast or other body parts using a handheld volumetric ultrasound probe; and miscellaneous volumetric imaging of the breast or other body parts using a robotic or other machine-driven volumetric ultrasound probe.

By way of further example, in yet another preferred embodiment, the texturably couplant-porous material sheet can be used in conjunction with a handheld linear array probe, although the amount of improvement in stability over the skin surface may be less dramatic since the relatively narrower footprint of such probes already provides appreciable stability. Still, there are several applications of one-dimensional handheld linear array probes that can benefit from such increased stability, particularly as combined with a corresponding ability to be moved when desired with only marginally greater effort than when the probe is smooth-surfaced. Examples include intercostal phased array transducers that may be used for cardiac imaging. Most transducers have a silicone lens covering that is the patient contact surface, this surface becoming particularly slippery when coated with coupling gel, and many applications using such transducers can benefit from such increased stability, especially when image quality is not compromised (or, in some cases, is even improved) by the texturably couplant-porous material sheet. For the one-dimensional array context, it has been found especially advantageous to use a polyester organza material having a filament diameter of about 40 microns and a filament spacing of about 500 microns in conjunction with a handheld linear array probe.

By way of even further example, while described and/or illustrated as covering the entire two-dimensional scan area in one or more of the preferred embodiments supra, it is also within the scope of the preferred embodiments for the texturably couplant-porous material sheet to only cover part of the two-dimensional scan area, such as in discrete strips, squares, or circular sub-areas, to even further minimize effects on image quality while still providing for improved probe stability over the skin surface. By way of even further example, in yet another preferred embodiment, the texturably couplant-porous material sheet is used in conjunction with a handheld volumetric ultrasound transducer in which the cap (see elements 314, 514, supra) is made much thinner than on conventional wobbler probes. According to a preferred embodiment, a thinner cap is used so as to reduce artifacts that can be brought about by the conventionally thicker cap used on prior art probes. In one preferred embodiment, the thickness of the cap is reduced even to a point where it is substantially flexible and perhaps even sheet-like. Advantageously, whereas making a thinner cap could otherwise have brought about stability problems (e.g., increased slippage because the cap is flexing, or other stability problems), the use of a texturably couplant-porous material sheet in conjunction therewith according to one or more of the preferred embodiments supra can substantially offset and/or entirely obviate such concerns. Thus, reference to the details of the described embodiments are not intended to limit their scope, which is limited only by the scope of the claims set forth below.

What is claimed is:

1. An apparatus for ultrasonically scanning a tissue volume, comprising:
a handheld volumetric ultrasound scanning probe characterized by a two-dimensional scan area and having a substantially rigid cap that extends across said two-dimensional scan area; and
a texturably couplant-porous material sheet covering said substantially rigid cap over at least a portion of said two-dimensional scan area for facilitating positional stability of said handheld volumetric ultrasound scanning probe while positioned against a skin surface of the tissue volume.

2. The apparatus of claim 1, wherein said texturably couplant-porous material sheet comprises a material selected from the group consisting of: polyester organza materials, polyester chiffon fabrics, and cloth fabrics.

3. The apparatus of claim 1, wherein said two-dimensional scan area is at least about 2 cm×2 cm in size and wherein texturably couplant-porous material sheet covers said substantially rigid cap over all of said two-dimensional scan area.

4. The apparatus of claim 1, wherein said texturably couplant-porous material sheet is adhesively affixed to said substantially rigid cap.

5. The apparatus of claim 4, wherein said texturably couplant-porous material sheet is configured to be user-affixable to said substantially rigid cap and user-removable from said substantially rigid cap.

6. The apparatus of claim 5, wherein said texturably couplant-porous material sheet is disposable.

7. The apparatus of claim 1, wherein said handheld volumetric scanning probe is selected from the group consisting of: wobbler probes; probes having a linear array transducer that is mechanically actuated to cover said two-dimensional scan area; and probes having stationary two-dimensional solid-state transducers.

8. The apparatus of claim 1, further comprising:
a processor that (i) receives first ultrasonic scans of the tissue volume in a first state of compression from said handheld volumetric ultrasound scanning probe, (ii) receives second ultrasonic scans of the tissue volume in a second state of compression from said handheld volumetric ultrasound scanning probe different than said first state of compression, and (iii) processes said first and second ultrasonic scans to identify a potential lesion in said tissue volume that is exhibiting different elasticity characteristics in comparison to surrounding tissue; and
an output display coupled to said processor, said output display displaying at least one ultrasonic image of the potential lesion and the surrounding tissue in a manner that visually highlights in colorized format the different elasticity characteristics of said potential lesion relative to said surrounding tissue.

9. The apparatus of claim 8, wherein said output display displays a first ultrasonic image in composited colorized contrast with a second ultrasonic image, wherein said first ultrasonic image contains said potential lesion and said surrounding tissue in said second state of compression, and wherein said second ultrasonic image contains a hypothetical version of said potential lesion that would appear if the potential lesion had a same elasticity characteristic as said surrounding tissue while the state of compression was changed from said first state to said second state.

10. The apparatus of claim 8, wherein said output display is integral with a casing of said handheld volumetric ultrasound scanning probe.

11. A method for ultrasound examination of a compressible tissue volume, comprising:
providing a handheld volumetric ultrasound scanning probe having a cap and a two-dimensional scan area thereacross, wherein the handheld volumetric ultrasound scanning probe is provided with a texturably couplant-porous material sheet at least partially covering the two-dimensional scan area of the cap; and
operating the handheld volumetric ultrasound scanning probe to acquire ultrasonic scans of the tissue volume, including hand-manipulating the handheld volumetric ultrasound scanning probe to bring the two-dimensional scan area of the cap, as at least partially covered by the texturably couplant-porous material sheet, into contact with a skin surface of the tissue volume, the texturably couplant-porous material sheet facilitating positional stability of the handheld volumetric ultrasound scanning probe while so positioned against the skin surface.

12. The method of claim 11, said texturably couplant-porous material sheet comprising a material selected from the group consisting of polyester organza materials, polyester chiffon fabrics, and cloth fabrics, said cap being substantially rigid, said two-dimensional scan area being at least about 2 cm×2 cm in size, and said texturably couplant-porous material sheet covering said substantially rigid cap over all of said two-dimensional scan area.

13. The method of claim 11, said texturably couplant-porous material sheet being disposable, the method further comprising:
prior to said operating, covering the two-dimensional scan area of the cap with the texturably couplant-porous material sheet; and
subsequent to said operating, removing the texturably couplant-porous material sheet from the cap and disposing of texturably couplant-porous material sheet.

14. The method of claim 11, wherein said handheld volumetric scanning probe is selected from the group consisting of: wobbler probes; probes having a linear array transducer that is mechanically actuated to cover said two-dimensional scan area; and probes having stationary two-dimensional solid-state transducers.

15. The method of claim 11, wherein said operating the handheld volumetric ultrasound scanning probe further comprises:
bringing, by hand-manipulation, the handheld volumetric ultrasound scanning probe into a first state of compression against the skin surface to acquire first ultrasonic scans of the tissue volume;
bringing, by hand-manipulation, the handheld volumetric ultrasound scanning probe into a second state of compression against the skin surface to acquire second ultrasonic scans of the tissue volume, the second state of compression being different than the first state of compression, wherein a processor associated with the handheld volumetric ultrasound scanning probe processes said first and second ultrasonic scans to identify a potential lesion in the tissue volume that is exhibiting different elasticity characteristics in comparison to surrounding tissue; and
viewing, on an output display that is integral with the handheld volumetric ultrasound scanning probe, at least one ultrasonic image that visually highlights in colorized format the different elasticity characteristics of the potential lesion relative to the surrounding tissue.

16. The method of claim 15, wherein said output display displays a first ultrasonic image in composited colorized contrast with a second ultrasonic image, wherein said first ultrasonic image contains said potential lesion and said surrounding tissue in said second state of compression, and wherein said second ultrasonic image contains a hypothetical version of said potential lesion that would appear if the potential lesion had a same elasticity characteristic as said surrounding tissue while the state of compression was changed from said first state to said second state.

17. A system for ultrasonic examination of a compressible tissue volume, comprising:
a handheld volumetric ultrasound scanning probe characterized by a two-dimensional scan area and having a substantially rigid cap that extends across said two-dimensional scan area;
a texturably couplant-porous material sheet covering said substantially rigid cap over at least a portion of said two-dimensional scan area for facilitating positional stability of said handheld volumetric ultrasound scanning probe while positioned against a skin surface of the tissue volume;
a processor operatively coupled to said handheld volumetric ultrasound scanning probe, said processor (i) receiving first ultrasonic scans from the handheld volumetric ultrasound scanning probe while in a first state of compression against the skin surface, (ii) receiving second ultrasonic scans from the handheld volumetric ultrasound scanning probe while in a second state of compression against the skin surface different than said first state of compression, and (iii) processing said first and second ultrasonic scans to identify a potential lesion in said tissue volume that is exhibiting different elasticity characteristics in comparison to surrounding tissue; and
an output display coupled to said processor, said output display displaying at least one ultrasonic image of the potential lesion and the surrounding tissue in a manner that visually highlights in colorized format the different elasticity characteristics of said potential lesion relative to said surrounding tissue.

18. The system of claim 17, wherein said output display displays a first ultrasonic image in composited colorized contrast with a second ultrasonic image, wherein said first ultrasonic image contains said potential lesion and said surrounding tissue in said second state of compression, and wherein said second ultrasonic image contains a hypothetical version of said potential lesion that would appear if the potential lesion had a same elasticity characteristic as said surrounding tissue while the state of compression was changed from said first state to said second state.

19. The system of claim 17, said texturably couplant-porous material sheet comprising a material selected from the group consisting of polyester organza materials, polyester chiffon fabrics, and cloth fabrics, said two-dimensional scan area being at least about 2 cm×2 cm in size, and said texturably couplant-porous material sheet covering said substantially rigid cap over substantially all of said two-dimensional scan area.

20. The system of claim 17, wherein said texturably couplant-porous material sheet is provided as a disposable item for removal and disposal after each ultrasonic examination.
